(19) United States
(12) Patent Application Publication
(10) Pub. No.: US 2011/0301455 A1
(43) Pub. Date: Dec. 8, 2011

(54) OPTICAL TOMOGRAPHIC IMAGING APPARATUS

(75) Inventors: Yasuyuki Numajiri, Kawasaki-shi (JP); Kazuro Yamada, Kawasaki-shi (JP); Futoshi Hirose, Yokohama-shi (JP)

(73) Assignee: CANON KABUSHIKI KAISHA, Tokyo (JP)

(21) Appl. No.: 13/201,724

(22) PCT Filed: Feb. 24, 2010
(86) PCT No.: PCT/JP2010/053376

§ 371 Date: Aug. 16, 2011

(30) Foreign Application Priority Data
Mar. 6, 2009 (JP) 2009-052879

(51) Int. Cl. A61B 6/00

(52) U.S. Cl. 600/425

(57) ABSTRACT

Provided is an optical tomographic imaging apparatus that, at imaging a tomographic image in an OCT system with a high lateral resolution, can more reduce blurring in the image due to movement of an object, including: a scanning device for scanning, on the object, a first irradiation beam having a large spot diameter and a second irradiation beam having a small spot diameter synchronized with each other, an image information acquiring device for acquiring first and second image information obtained with the irradiation beams, respectively, by scanning the first and second irradiation beams, and a position correcting device for identifying a position of the first image information based on reference image information acquired in advance, and, based on correlation of a positional relation between the first and second image information, correcting a position of the second image information by associating the position with the identified first image information's position.

### TECHNICAL FIELD

The present invention relates to an optical tomographic imaging apparatus, and particularly to an optical tomographic imaging apparatus used for ophthalmologic diagnosis and treatment.

### BACKGROUND ART

An optical tomographic imaging apparatus can provide a high-resolution tomographic image of an object by Optical Coherence Tomography (OCT) using an interference phenomenon of multi-wavelength light. This apparatus has been occupying a place essential to imaging a tomographic image of the retina in the ophthalmologic field. Such an optical tomographic imaging apparatus using an OCT system will be hereinafter called “an OCT apparatus”. The OCT apparatus described above can irradiate a measuring beam of low-coherence light on an object and measure backscattering light from the object with high sensitivity by using an interferometer. Moreover, scanning the measuring beam on the object can provide a tomographic image with high resolution.

Recently, an OCT apparatus for ophthalmology is shifting from a conventional time-domain system to a Fourier-domain system capable of imaging at a higher speed. This Fourier-domain system includes a spectral-domain system that separates coherent light into spectral components, and a swept source system using a light source capable of wavelength scanning. Also, imaging with a higher resolution has been tried, but because movement of the eyeball has a greater effect on blurring or deletion in an image, even imaging at a high speed by the Fourier-domain system has not satisfactorily solved the problems yet. In ophthalmologic equipment, to reduce various effects due to this movement of the eyeball, the movement of the eyeball, until now, has been detected to track the movement.

Also, in an ophthalmologic OCT apparatus, Japanese Patent Publication No. 3976678 proposes a tracking system in which reflection of a tracking beam is analyzed to detect movement of the eyeball and a scanning beam for OCT is controlled to follow the movement.

On the other hand, an OCT apparatus executes imaging and measurement, so that after taking data of an image or measurements, the data can also be corrected. The pamphlet of International Publication No. 2007/039267 discloses an OCT apparatus that corrects data in the following manner. That is, in the OCT apparatus, a position of a cornea surface is measured by a first OCT using the time-domain system or the Fourier-domain system, an eye axis length is measured by a second, similar OCT, and a measurement error in the eye axis length measured by the second OCT due to movement of the eyeball is corrected by using a positional measurement by the first OCT.

### DISCLOSURE OF THE INVENTION

An OCT apparatus with a high lateral resolution has a shallow depth of focus, so that it becomes necessary to correct a position for a tomographic image to be obtained by OCT (OCT image) to reduce blurring in the tomographic image due to movement of an object such as the eyeball. However, if the OCT apparatus in conventional examples described above, which includes a method for correcting a position in such a manner, has a high lateral resolution, it has not been necessarily achieved satisfactorily to reduce blurring in the tomographic image due to the movement of the object such as the eyeball.

In view of the problems described above, an object of the present invention, for imaging a tomographic image by an OCT system with a high lateral resolution, is to provide an optical tomographic imaging apparatus which can more reduce blurring in an image due to movement of the object.

The present invention provides an optical tomographic imaging apparatus constructed in the following manner. The optical tomographic imaging apparatus according to the present invention is an optical tomographic imaging apparatus for imaging a tomographic image of an object using optical coherence tomography (OCT), characterized by including:

a scanning device for scanning, on the object, a first irradiation beam having a large spot diameter and a second irradiation beam having a small spot diameter which are synchronized with each other,

an image information acquiring device for acquiring first image information obtained with the first irradiation beam and second image information obtained with the second irradiation beam by scanning the first and second irradiation beams using the scanning device, and

a position correcting device for identifying a position of the first image information based on reference image information, and correcting a position of the second image information by associating the position with the identified position of the first image information based on correlation of a positional relation provided by the synchronized scan between the first image information and the second image information.

The present invention can realize an optical tomographic imaging apparatus which, at imaging a tomographic image by an OCT system with a high lateral resolution, can more reduce blurring in an image due to movement of an object.

Further features of the present invention will become apparent from the following description of exemplary embodiments with reference to the attached drawings.

### BRIEF DESCRIPTION OF THE DRAWINGS

FIG. 1 illustrates a configuration of an optical system in an OCT apparatus of an embodiment 1 of the present invention.

FIG. 2A illustrates optical paths on the side of outgoing beams in the OCT apparatus of the embodiment 1 of the present invention.

FIG. 2B illustrates incoming of a measuring beam into the eye to be inspected from the OCT apparatus.

FIG. 2C illustrates optical paths of added light beams in the OCT apparatus.

FIG. 3 illustrates a scan pattern in an image forming apparatus in the OCT apparatus of the embodiment 1 of the present invention.

FIG. 4 illustrates a procedure flow for position correction in the image forming apparatus in the OCT apparatus of the embodiment 1 of the present invention;

FIG. 5A illustrates a scan pattern in the image forming apparatus in the OCT apparatus of the embodiment 1 of the present invention.

FIGS. 5B and 5C illustrate position determination of A scan information in the image forming apparatus in the OCT apparatus of the embodiment 1 of the present invention.

FIG. 6A illustrates a scan pattern in an image forming apparatus in an OCT apparatus of an embodiment 2 of the present invention.

FIG. 6B illustrates a procedure flow for position correction in the image forming apparatus in the OCT apparatus of the embodiment 2 of the present invention.

FIG. 7A illustrates a configuration of an optical system in an OCT apparatus of an embodiment 3 of the present invention.

FIG. 7B illustrates optical paths on the side of outgoing beams in the OCT apparatus of the embodiment 3 of the present invention.

FIG. 8A illustrates a procedure flow for position correction in an image forming apparatus in the OCT apparatus of the embodiment 3 of the present invention.

FIG. 8B illustrates a scan pattern in the image forming apparatus in the OCT apparatus of the embodiment 3 of the present invention.

### BEST MODES FOR CARRYING OUT THE INVENTION

Best modes for carrying out the present invention will be described with reference to the following embodiments.

### EMBODIMENTS

Embodiments of the present invention will be hereinafter described.

### Embodiment 1

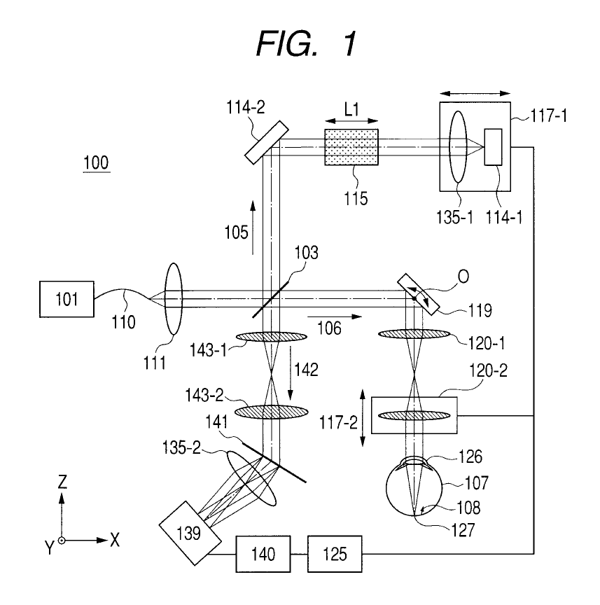

In an embodiment 1, an optical tomographic imaging apparatus according to the present invention using an OCT system will be described. Particularly, here, an optical tomographic imaging apparatus whose object is the eye to be inspected is described. First, an outline of an overall configuration of an optical system in the optical tomographic imaging apparatus of this embodiment is described with reference to FIG. 1. Note that, regarding a direction of the eye to be inspected 107 in FIG. 1, an upward direction thereof is oriented in the plus Y direction, and a downward direction in the minus Y direction of the XYZ axes shown.

An optical tomographic imaging apparatus 100 of this embodiment using an OCT system (hereinafter, called “an OCT apparatus 100”) is an OCT apparatus using the spectral-domain system, which separates coherent light into spectral components, of the Fourier-domain system. Also, the OCT apparatus 100 of this embodiment, as shown in FIG. 1, forms a Michelson interferometer as a whole. In FIG. 1, light emitted from a light source 101 is split into a reference beam 105 and a measuring beam 106 by a beam splitter 103. The measuring beam 106 is reflected or scattered by the eye to be inspected 107, which is the object of observation, to provide a return beam 108 which comes back and is added to the reference beam 105 by the beam splitter 103. The reference beam 105 and the return beam 108, after the addition, are separated into wavelengths by a transmissive grating 141, and then enter a line camera 139. The line camera 139 converts light intensity into a voltage for each of positions (wavelengths), and the resultant signals are used to form a tomographic image of the eye to be inspected 107.

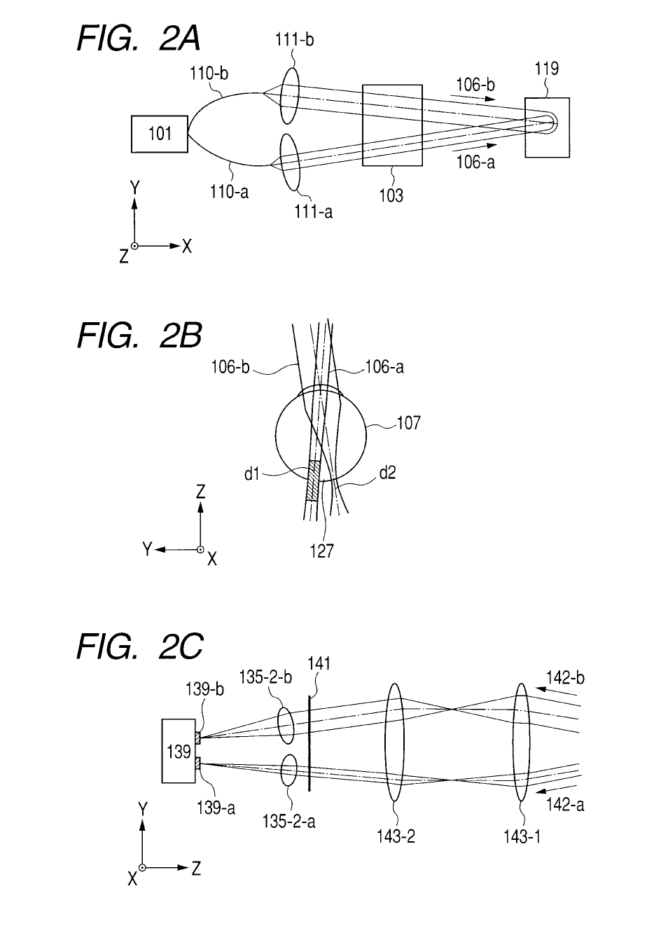

Next, details of the light source 101 are described. The light source 101 is a super luminescent diode (SLD) that is a representative, low-coherence light source. The light source 101 has a wavelength of 830 nm and a bandwidth of 50 nm. The bandwidth, here, is a very important parameter because it has the effect on resolution of a tomographic image to be obtained in an optical axis direction. Also, SLD, here, has been selected as a type of the light source, but any light sources that can emit low-coherence light may be used, and Amplified Spontaneous Emission (ASE) may be also used. Also, because an eye is measured, wavelengths of near infrared light are appropriate. Further, because wavelengths have the effect on resolution in a lateral direction of a tomographic image to be obtained, wavelengths are desirably as shorter as possible, and here, the wavelength of 830 nm has been selected. Any other wavelengths may be selected depending on a measured position of an object to be observed. Light emitted from the light source 101 is directed to a lens 111 through two single-mode fibers 110. FIG. 2A illustrates, in the XY plane, optical paths on the side of outgoing beams of these two single-mode fibers 110-a, and 110-b. The light emitted from the two single-mode fibers 110-a, and 110-b is adjusted by lenses 111-a, and 111-b to be a first beam having a small beam diameter (beam diameter: 1 mm) and a second beam having a large beam diameter (beam diameter: 4 mm) that arecollimated, respectively. These beams, then, go to the beam splitter 103.

Next, an optical path of the reference beam 105 is described. The reference beam 105 split by the beam splitter 103 enters a mirror 114-2 to change its direction, and it, then, is condensed by a lens 135-1 on a mirror 114-1 and reflected by the mirror 114-1 to come back to the beam splitter 103. Next, the reference beam 105 passes through the beam splitter 103 and is directed to the line camera 139. A glass 115 is used for dispersion compensation. The glass 115 for dispersion compensation compensates dispersion of the measuring beam 106 when it goes to and comes back from the eye to be inspected 107 for the reference beam 105. Here, a representative value of a diameter of the Japanese average eyeball is assumed to be L1=23 mm. Moreover, an electrically-driven stage 117-1 can move to directions shown by the arrows, and adjust and control an optical path length of the reference beam 105.

Next, an optical path of the measuring beam 106 is described. The measuring beam 106 split by the beam splitter 103 enters a mirror of an XY scanner 119. Here, for the simplicity, the XY scanner 119 is shown as one mirror, but in fact, two mirrors for X scanning and Y scanning are placed close to each other, respectively and scan the retina 127 in a direction perpendicular to an optical axis in the raster scan mode. As illustrated in FIG. 2A, the light emitted from the two single-mode fibers 110-a, and 110-b provides measuring beams 106-a, and 106-b, respectively. Also, the center of the measuring beam 106 is adjusted to correspond with the rotation center O of the mirror of the XY scanner 119. Lenses 120-1 and 120-2 form an optical system for scanning the retina 127, and have the same magnification. The lenses 120-1 and 120-2 have a role in scanning the measuring beam 106 on the retina 127 by using an area around the cornea 126 as apupil of the optical system. Moreover, an electrically-driven stage is shown by the reference number 117-2, and it can move to directions shown by the arrows, and adjust and control a position of the associated lens 120-2. Adjusting the position of the lens 120-2 allows the measuring beam 106 to be condensed on a desired layer of the retina 127 in the eye to be inspected 107 to observe. Further, it is also possible to address the case where the eye to be inspected 107 has a refractive error.

FIG. 2B illustrates incoming of the measuring beam into the eye to be inspected 107. The light emitted from the two single-mode fibers 110-a, and 110-b, as shown in FIG. 2B, is condensed on the retina with spot diameters being d1 and d2 according to the following equation, respectively.

d=4λ·f/(π·ε) (1)

Where, d is the spot diameter, λ is a wavelength, which is 830 nm in this embodiment, and f is a focal length of the eye to be inspected 107. Also, ε is a beam diameter when the light enters the lens 120-1. The beam diameters of the measuring beams 106-a, and 106-b provided by the light emitted from the two single-mode fibers 110-a, and 110-b, in this embodiment, are adapted to be 1 mm and 4 mm, respectively. And, the spot diameter d is inversely proportional to the beam diameter ε according to the equation (1). Therefore, the spot diameter d1 of the measuring beam 106-a having the beam diameter of 1 mm forms a large spot diameter, and the spot diameter d2 of the measuring beam 106-b having the beam diameter of 4 mm forms a small spot diameter. The spot diameters d1 and d2 slightly vary depending on a focal length of the eye to be inspected 107 and the position of the lens 120-2. In this embodiment, they are approximately 20 μm and 5 μm, respectively. Once the measuring beam 106 enters the eye to be inspected 107, the measuring beam 106 provides the return beam 108 due to reflecting off or scattering from the retina 127, which is reflected by the beam splitter 103 to be directed to the line camera 139. Here, the electrically-driven stage 117-2 is adapted to be controlled by a personal computer 125.

Next, a configuration of a measuring system in the OCT apparatus of this embodiment is described. The OCT apparatus 100 can provide a tomographic image (OCT image) formed of intensity of an interference signal acquired by a Michelson interferometer. That measuring system is as follows. The return beam 108 that is the light reflected by or scattered from the retina 127 is reflected by the beam splitter 103. Here, the reference beam 105 and the return beam 108 are adjusted to be added to each other at the back of the beam splitter 103. Then, light 142 resulting from the addition passes through lenses 143-1 and 143-2 and enters the transmissive grating 141. The light 142, then, is separated into wavelengths by the transmissive grating 141 and subsequently condensed by a lens 135-2, and light intensity is converted into a voltage for each of positions (wavelengths) by the line camera 139.

FIG. 2C illustrates, in the YZ plane, an optical path of the added light 142 leading to the line camera 139. In this embodiment, the line camera 139 used is a type having a plurality of sensor portions, and two sensors 139-a, and 139-b of them are used. And, the light 142 formed from the addition of the light emitted from the two single-mode fibers 110-a, and 110-b is called light beams 142-a, and 142-b, respectively. These light beams pass through common lenses 143-1 and 143-2 to be again collimated light beams, which are separated into wavelengths by the transmissive grating 141. Subsequently, these light beams are condensed by lenses 135-2-a, and 135-2-b, respectively, and received by individual, different sensors 139-a, and 139-b of the line camera 139, respectively. Specifically, interference bands in a spectral region in a wavelength axis will be observed on the line camera 139.

Note that depths of focus (DOF) of the irradiation beams having the spot diameter d1 (large spot diameter, here 20 μm) and the spot diameter d2 (small spot diameter, here 5 μm), respectively, are approximately 1 mm and 60 μm, respectively, according to the following equation (2) and further with consideration for a refractive index.

DOF=π·d²/(2λ) (2)

The resultant group of voltage signals is converted into a digital value by a frame grabber 140 and data processed by the personal computer 125 to form a tomographic image. Here, the sensors 139-a, and 139-b of the line camera 139 have 1024 pixels, respectively and can provide the intensity of the added light 142 for each of wavelengths (1024 divisions).

Next, a device for acquiring a tomographic image in the OCT system of this embodiment (image information acquiring device) is described. The OCT apparatus 100, which includes a controlling device for controlling the XY scanner 119 (not shown), can acquire interference bands by the line camera 139 by controlling the controlling device, acquiring a tomographic image of the retina 127 (FIG. 1). Once the measuring beam 106 enters the retina 127 through the cornea 126, it provides the return beam 108 due to reflection or scattering at various positions, which arrives at the line camera 139 with time delays at various positions. Here, the light source 101 has a wide bandwidth and a short spatial coherence length, and when an optical path length of the reference beam path is approximately equal to an optical path length of the measuring beam path, the interference bands can be observed on the line camera 139. As described above, what are acquired by the line camera 139 are the interference bands in a spectral region in a wavelength axis. Next, the interference bands, which provide information in the wavelength axis, are converted into interference bands in the optical frequency axis with consideration for characteristics of the line camera 139 and the transmissive grating 141. Further, inverse Fourier transforming the converted interference bands in the optical frequency axis can provide information in a depth direction (so called “A scan information”). Then, detecting the interference bands while driving an X axis of the XY scanner 119 can provide interference bands at each of positions in each of the X axes. That is, information at each of positions in each of the X axes in the depth direction (so called “B scan information”) can be acquired. As the result, two-dimensional distribution of the intensity of the return beam 108 in the XZ plane can be acquired, that is, it is a tomographic image.

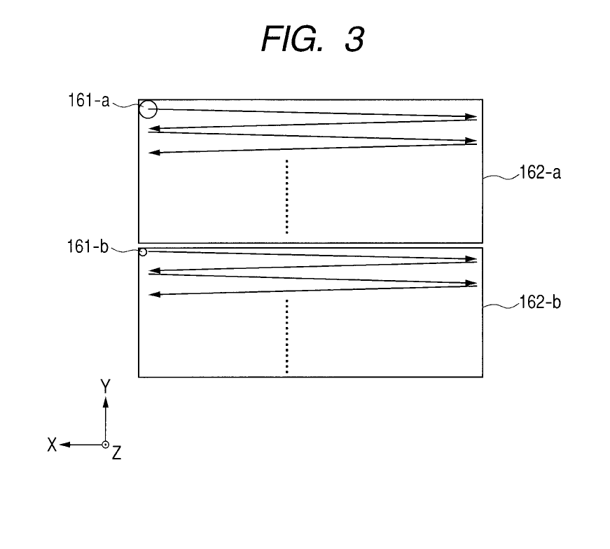

Next, position correction of tomographic image information in this embodiment is described. FIG. 3 illustrates a scan pattern of irradiation beams. In this embodiment, as shown in FIG. 3, a first irradiation beam 161-a having a spot diameter d1 (large spot diameter, here 20 μm) and a second irradiation beam 161-b having a spot diameter d2 (small spot diameter, here 5 μm), which are irradiated on the retina, are spaced apart from each other by about 200 μm. In such a manner, it is possible in this configuration to irradiate the first irradiation beam and the second irradiation beam closely to each other on an object through a device for controlling a scan of the XY scanner 119. The OCT apparatus 100 controls the XY scanner 119 to scan the first and second irradiation beams 161-a, and 161-b within scan ranges 162-a, and 162-b in directions shown by the arrows in FIG. 3 in the raster scan mode. At this time, the measuring beams 106-a, and 106-b are reflected by the common mirror of the XY scanner 119, so that the first irradiation beam 161-a and the second irradiation beam 161-b are synchronized with each other to scan.

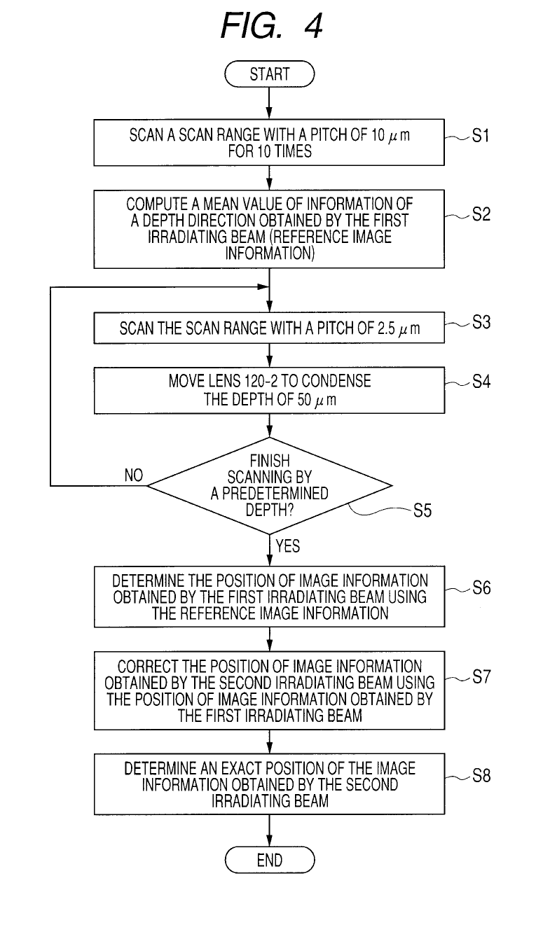

FIG. 4 illustrates a procedure flow for position correction. First, at step S1, a scan, as shown in FIG. 3, is carried out with a pitch of about 10 μm per line in the Y direction. Then, after the scan ranges 162-a, and 162-b are entirely scanned, the XY scanner 119 is controlled to come back to scan start positions in the scan ranges 162-a, and 162-b. Then, the scan ranges 162-a, and 162-b are entirely scanned again, and a scan is repeated for 10 times in total. Next, at step S2, information in the depth direction is acquired at each position in the XY plane (lateral direction) in the scan range 162-a scanned at the step S1 by the first irradiation beam 161-a. Here, a mean value of ten scans is computed for each pixel of each piece of such A scan information. Then, data different from the mean valve by equal to or more than the standard deviation is removed, and a mean value is computed again using only the data within the standard deviation. Note that the data different by equal to or more than the standard deviation may be considered to be generated by a large movement or a blink of the eye to be inspected 107. The entire information in the depth direction (Z direction) in the lateral direction (in the XY plane) using this mean value is called a reference image in the scan range 162-a for position correction (reference image information). In such a manner, the reference image information is acquired in advance in this embodiment and stored in the personal computer 125.

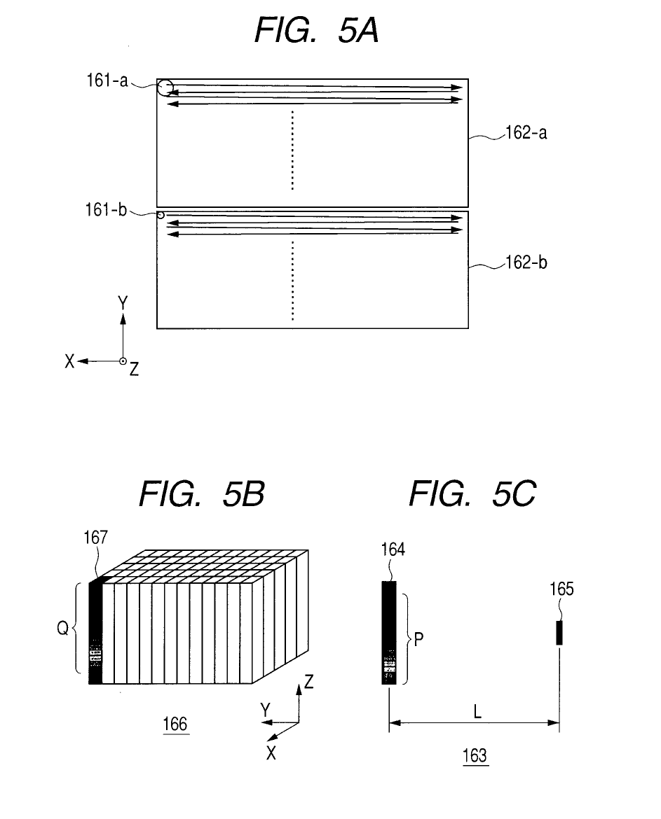

At step S3, the scan ranges 162-a, 162-b are entirely scanned with a scan pitch of about 2.5 μm per line in the Y direction this time. FIG. 5A illustrates a scan pattern of the irradiation beams at the step S3. Next, at step S4, the electrically-driven stage 117-2 is moved, that is, the lens 120-2 is moved to condense the measuring beam 106 at a deep position of about 50 μm in the retina. At step S5, it is determined whether a scan at a predetermined depth is finished or not, and these steps S3 and S4 are repeated until a scan at a predetermined depth is finished. Because the eye to be inspected 107 usually moves during the scans, simply arranging the resultant image information results in a distorted image. Therefore, at step S6, a position of each piece of the A scan information (first image information) obtained by the first irradiation beam 161-a of the image information obtained at the step S3 is determined in the depth direction corresponding to the Z direction and in the lateral direction corresponding to the XY directions of the XYZ coordinate system.

FIGS. 5B and 5C conceptually illustrates a method for determining a position of one piece of the A scan information in the lateral direction and in the depth direction. In FIG. 5C, One example of the A scan information 165 obtained by the second irradiation beam 161-b corresponding to the A scan information 164 obtained by the first irradiation beam 161-a, is shown by the reference number 163. The A scan information 164 and 165 illustrates, by gradation, the results from analysis of light intensity in the depth direction, respectively, and larger intensity is shown darker. A relative distance between an A scan position scanned by the first irradiation beam 161-a, and an A scan position scanned by the second corresponding irradiation beam 161-b is shown by the reference symbol L, and the relative distance is approximately 200 μm in this embodiment. In FIG. 5B, The reference image information is shown by the reference number 166, and 5 pieces of the A scan information in the X direction and 13 pieces of the A scan information in the Y direction of the reference image information obtained at the step S2 are representatively arrayed in a cubic form. The retina is shown flat without consideration for its curvature. Also, a scan direction of one line is adapted to be parallel to the X axis direction. One piece of the A scan information of the reference image information is representatively shown by the reference number 167, and the result from analysis of the light intensity in the depth direction is shown by gradation. For any other A scan information, illustration of the light intensity in the depth direction like 167 is omitted. To determine a position of each piece of the A scan information (first image information) obtained by the first irradiation beam 161-a in the lateral direction and in the depth direction, the image information obtained by the first irradiation beam 161-a is compared to the reference image information obtained at the step S2. Specifically, a light intensity pattern (gradation pattern) of one piece of the A scan information 164 obtained at the step S3 is pattern matched with all light intensity patterns of the reference image information 166 using a correlation function. Then, A scan information that most corresponds to the A scan information 164 is acquired. At this time, pattern-matching also in the depth direction is carried out to acquire a position that most corresponds in the XYZ coordinate system, and a position of the A scan information 164 is identified. In FIGS. 5B and 5C, because a P part of the A scan information 164 corresponds to a Q part of the A scan information 167, a position can be identified. This step is applied to all the A scan information.

Next, at step S7, based on the position determination result of the A scan information by the first irradiation beam 161-a at the step S6, a position of the A scan information (second image information) obtained by the second irradiation beam 161-b is corrected. Because the first irradiation beam 161-a, and the second irradiation beam 161-b are synchronized with each other to scan, a relative positional relation between them is always constant. Therefore, once the position of the A scan information (first image information) by the first irradiation beam 161-a is determined, a position of the corresponding A scan information (second image information) by the second irradiation beam 161-b may accordingly be aligned therewith. This step is applied to all the A scan information by the second irradiation beam 161-b.

By the way, at the step S7, to align the position of the A scan information by the second irradiation beam 161-b, the information by the first irradiation beam 161-a, i.e. information having the large spot diameter and a low lateral resolution is used as a basis. Therefore, a position to be determined will have a low lateral resolution. Then, to determine an exact position of the corresponding A scan information by the second irradiation beam 161-b, the following processes are carried out. First, at determining a position of one piece of the A scan information by the first irradiation beam 161-a at the step S6, an exact position is determined as follows when there is no movement in the lateral direction (XY directions) and a scan position corresponds to a position of information. That is, in the correction at the step S7 as described above, each position of the corresponding A scan information by the second irradiation beam 161-b is arranged in scan order to determine each exact position. Then, if there is movement in the lateral direction (XY directions) and the scan position does not correspond to the position of the information, a plurality of pieces of the A scan information by the second irradiation beam 161-b will be usually assigned to that position. An exact position for that position is determined at step S8 so that adjacent pieces of the A scan information is closer to each other. Specifically, for one combination of exact positions of the a plurality of pieces of the A scan information, the sum of a correlation function between light intensity patterns of adjacent pieces of the A scan information is acquired. Then, for all the combinations, the sums thereof are acquired, and a combination having the highest value is adopted as closer adjacent pieces of the information.

As described above, the position correction of the tomographic image information corrects a distorted image due to movement of the eye to be inspected 107. Accordingly, in the OCT apparatus using the Fourier-domain system with a high lateral resolution, blurring in an image due to movement of the eyeball can be easily and more reduced without a complex tracking system. Particularly, in this embodiment, blurring in an image can be easily reduced in both of the depth direction and the lateral direction.

### Embodiment 2

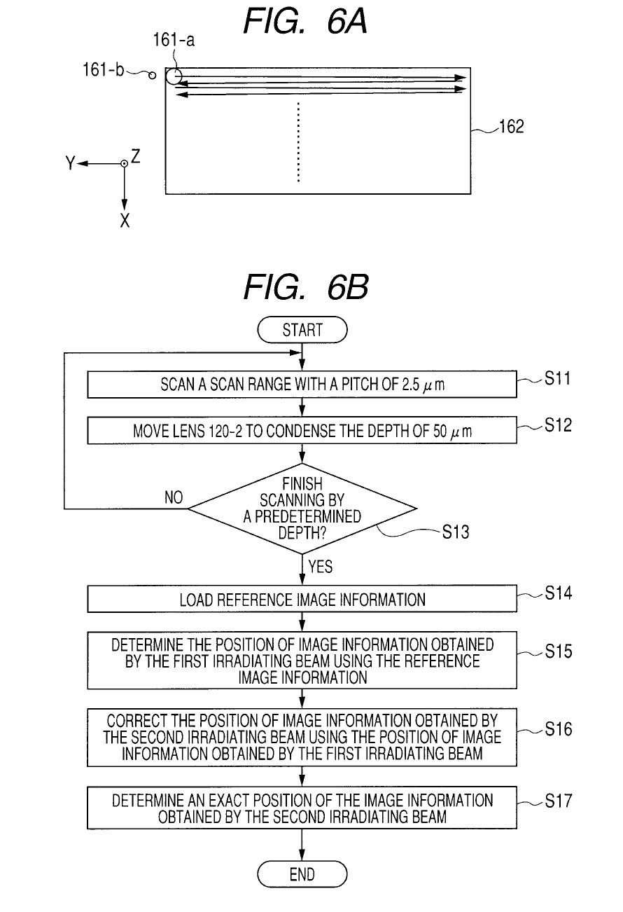

An OCT apparatus 100 of this embodiment is similar to that of the embodiment 1, and the rough configuration of the whole optical system shown in FIG. 1 can apply as is. However, in this embodiment, regarding a direction of the eye to be inspected 107 shown in FIG. 1, an upward direction is oriented in the minus X direction and a downward direction in the plus X direction of the XYZ axes shown. Next, position correction of tomographic image information in this embodiment is described. FIG. 6A illustrates a scan pattern of irradiation beams in this embodiment. A first irradiation beam 161-a having a spot diameter d1 (large spot diameter, here 20 μm) and a second irradiation beam 161-b having a spot diameter d2 (small spot diameter, here 5 μm), which are irradiated on the retina, are spaced apart from each other in a scan direction by about 25 μm. The OCT apparatus 100 of this embodiment controls and drives the XY scanner 119 to scan these first and second irradiation beams 161-a, and 161-b approximately within a scan range 162 in directions shown by the arrows in FIG. 6A in the raster scan mode. At this time, measuring beams 106-a, and 106-b are reflected by the common mirror of the XY scanner 119, and the first irradiation beam 161-a and the second irradiation beam 161-b are synchronized with each other to scan.

FIG. 6B illustrates a procedure flow for position correction. In this embodiment, reference image information used as a basis for alignment is acquired in advance and stored in the personal computer 125 similarly to the embodiment 1. A display position of an internal visual fixation light to change a direction of visual fixation of the eye to be inspected (not shown) and a scan position of a scanner are also stored. After the internal visual fixation light is lit at the display position of the internal visual fixation light stored, first, at step S11 as shown in FIG. 6A, a scan is carried out with a pitch of about 2.5 μm per line in the Y direction. Then, after a scan range 162 for the scan position stored is entirely scanned, the XY scanner 119 is controlled to come back to scan start positions in the scan range 162. Next, at step S12, the electrically-driven stage 117-2 is moved, that is, the lens 120-2 is moved to condense the measuring beam 106 at a deep position of about 50 μm in the retina. At step S13, it is determined whether a scan at a predetermined depth is finished or not, and these steps S11 and S12 are repeated until a scan at a predetermined depth is finished.

At step S14, the reference image information stored in the personal computer 125 is read in, and at step S15, a position of each piece of A scan information obtained by the first irradiation beam 161-a among the image information obtained at the step S11 is determined in the lateral direction and in the depth direction. Similarly to the embodiment 1, the image information by the first irradiation beam 161-a is compared to the reference image information read in at the step S14. Specifically, a light intensity pattern (gradation pattern) of one piece of the A scan information obtained at the step S11 is pattern matched with a light intensity pattern of the reference image information using a correlation function, and A scan information of the reference image information that most corresponds is acquired. At this time, pattern-matching in the depth direction is also carried out to acquire a position that most corresponds in the lateral direction and in the depth direction, and a position of one piece of the A scan is identified. This step is applied to all the A scan information. Next, at step S16, based on the position determination result of the A scan information by the first irradiation beam 161-a at the step S14, a position of the A scan information obtained by the second irradiation beam 161-b is corrected. Because the first irradiation beam 161-a, and the second irradiation beam 161-b are synchronized with each other to scan similarly to the embodiment 1, a relative positional relation between them is always constant. Therefore, once the position of the A scan information by the first irradiation beam 161-a is determined, a position of the corresponding A scan information by the second irradiation beam 161-b may accordingly be aligned therewith. This step is applied to all the A scan information by the second irradiation beam 161-b.

Here, similarly to the embodiment 1, at determining a position of one piece of the A scan information by the first irradiation beam 161-a at the step S15, if there is no movement in the lateral direction (XY directions) and a scan position corresponds to a position of information, an exact position is determined as follows. That is, in the correction at the step S16 as described above, each position of the corresponding A scan information by the second irradiation beam 161-b is arranged in scan order to determine each exact position. Then, if there is movement in the lateral direction (XY directions) and the scan position does not correspond to the position of the information, a plurality of pieces of the A scan information by the second irradiation beam 161-b will be usually assigned to that position. An exact position for that position is determined at step S17 so that adjacent pieces of the A scan information is closer to each other. Specifically, for one combination of exact positions of the a plurality of pieces of the A scan information, the sum of a correlation function between light intensity patterns of adjacent pieces of the A scan information is acquired. Then, for all the combinations, the sums thereof are acquired, and a combination having the highest value is adopted as closer adjacent pieces of the information.

As described above, the position correction of the tomographic image information corrects a distorted image due to movement of the eye to be inspected 107. Accordingly, also in the OCT apparatus using the Fourier-domain system with a high lateral resolution, blurring in an image due to movement of the eyeball can be easily and more reduced without a complex tracking system. Also in this embodiment, blurring in an image can be easily reduced in both of the depth direction and the lateral direction.

In this embodiment, the first irradiation beam 161-a, and the second irradiation beam 161-b are irradiated closely to each other. Therefore, a small difference in relative position aberration between the two irradiation beams caused by curvature of the retina to movement of the eye to be inspected 107 in the Z direction allows a distorted image to be corrected more accurately than the case where the two irradiation beams are spaced apart from each other. Also, in this embodiment, to reduce the amount of a crosstalk between the two beams, i.e. the first irradiation beam 161-a, and the second irradiation beam 161-b, their irradiation positions are not matched with each other, but are adjoined. However, their irradiation positions can be also matched with each other if two beams different in wavelength are used for wavelength separation. Also, in this embodiment, information used as a basis for alignment is obtained in advance as the reference image information, so that an imaging time becomes short and the load on a subject becomes small. Note that the reference image information, in this embodiment, is obtained in advance by the method of the embodiment 1, but the reference image information may be configured in advance by using another OCT apparatus and stored in the personal computer 125. Also, in this embodiment, the case has been described where the range in which the reference image information is stored and the scan range are matched with each other, but the scan range may be a part of the range in which the reference image information is stored. Alternatively, if the range in which the reference image information is acquired is sufficiently wide, it is not necessary to display the internal visual fixation light at a particular position, and the tomographic image information may be acquired by using an arbitrary position as the scan range.

### Embodiment 3

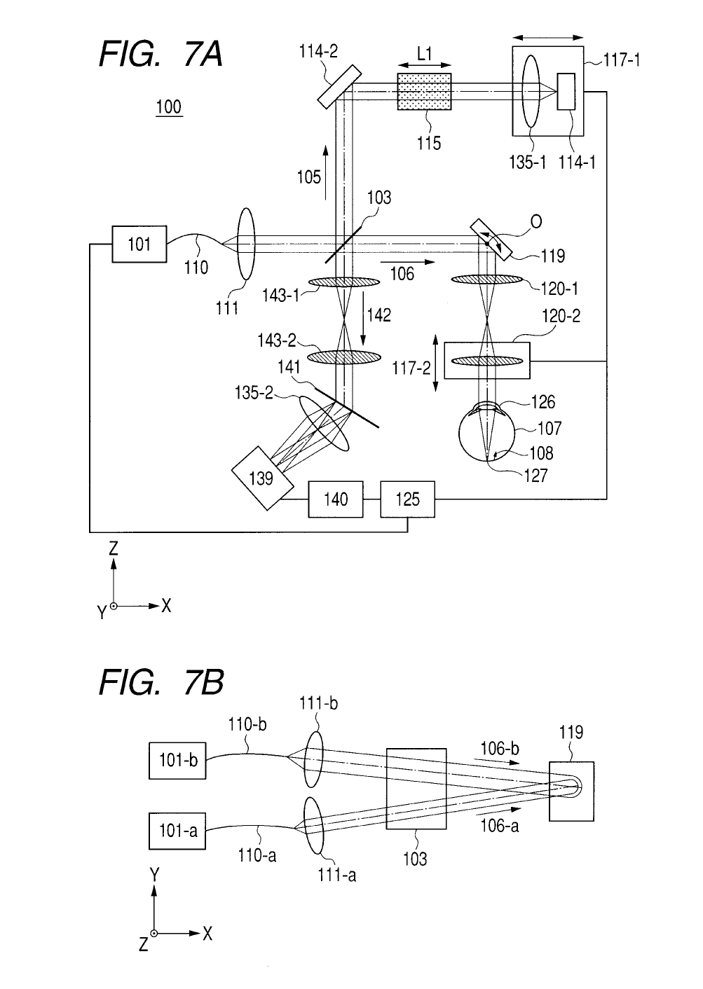

Next, an exemplary configuration of an OCT apparatus 100 in an embodiment 3 is described. As shown in FIG. 7A, a personal computer 125 is connected to a light source 101, and an on or off of the light source is adapted to be controlled by the personal computer 125. Any other configuration is similar to the embodiment 1, and description thereof is omitted. However, regarding a direction of the eye to be inspected 107 shown in FIG. 7A, similarly to the embodiment 2, an upward direction is oriented in the minus X direction and a downward direction in the plus X direction of the XYZ axes as shown. FIG. 7B, similarly to FIG. 2, illustrates optical paths on the side of outgoing beams of two single-mode fibers 110-a, and 110-b in the XY plane. In this embodiment, there are two light sources, and the two light source 101-a, and 101-b correspond to individual fibers, respectively. The outgoing beams of the two single-mode fibers 110-a, and 110-b are adjusted by lenses 111-a, and 111-b to be collimated beams having a beam diameter of 1 mm and a beam diameter of 4 mm, respectively, which go to the beam splitter 103.

Next, position correction of tomographic image information in this embodiment is described. Similarly to the embodiment 2, as shown in FIG. 6A, in this embodiment, a first irradiation beam and a second irradiation beam are spaced apart from each other as follows. That is, the first irradiation beam 161-a having a spot diameter d1 (large spot diameter, here 20 μm) and the second irradiation beam 161-b having a spot diameter d2 (small spot diameter, here 5 μm), which are irradiated on the retina, are spaced apart from each other in a scan direction by about 25 μm. The OCT apparatus 100 controls and drives the XY scanner 119 to scan these first and second irradiation beams 161-a, and 161-b approximately within a scan range 162 in directions shown by the arrows in FIG. 6A in the raster scan mode. At this time, measuring beams 106-a, and 106-b are reflected by the common mirror of the XY scanner 119, so that the first irradiation beam 161-a and the second irradiation beam 161-b are synchronized with each other to scan.

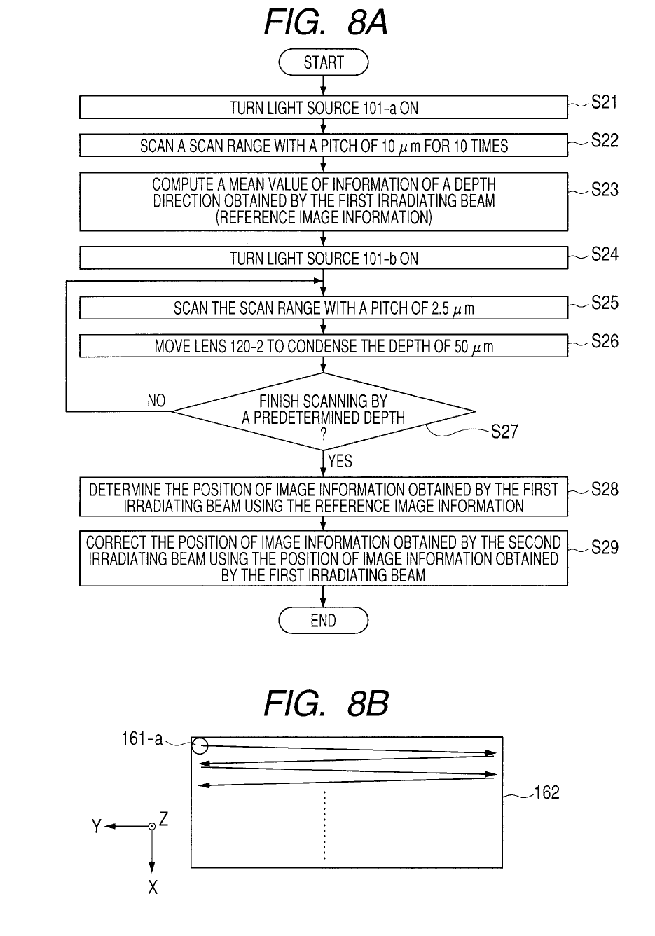

FIG. 8A illustrates a procedure flow for position correction. First, at step S21, only the light source 101-a is turned on. FIG. 8B illustrates, in this embodiment, a scan pattern of the irradiation beams. At step S22, a scan is carried out by using the first irradiation beam 161-a from the light source 101-a with a pitch of about 10 μm per line in the Y direction. After the scan range 162 is entirely scanned, the XY scanner 119 is controlled to come back to a scan start position in the scan ranges 162. Then, the scan range 162 is entirely scanned again, and a scan is repeated for 10 times in total.

Next, at step S23, regarding information at each position in the scan range 162 in the XY plane (lateral direction) obtained by the first irradiation beam 161-a at the step S22 as described above, i.e. regarding information about each pixel of each piece of A scan information, a mean value of ten scans is computed. Then, data different from the mean value by equal to or more than the standard deviation is removed, and a mean value is computed again using only the data within the standard deviation. Note that the data different by equal to or more than the standard deviation may be considered to be generated by a large movement or a blink of the eye to be inspected 107. The entire information in a lateral direction (in the XY plane) and in a depth direction (Z direction) in the scan range 162 using this mean value is used as a reference image (reference image information) for position correction. Then, at step S24, the light source 101-b is also turned on.

Next, at step S25, as shown in FIG. 6A, a scan is carried out by using both of the first and second irradiation beams 161-a, and 161-b this time with a pitch of about 2.5 μm per line in the Y direction, and the scan range 162 is entirely scanned. Further, at step S26, the electrically-driven stage 117-2 is moved, that is, the lens 120-2 is moved to condense the measuring beam 106 at a deep position of about 50 μm in the retina. At step S27, it is determined whether a scan at a predetermined depth is finished or not, and these steps S25 and S26 are repeated until a scan at a predetermined depth is finished. At step S28, a position of each piece of the A scan information by the first irradiation beam 161-a of the image information obtained at the step S25 is determined in the depth direction. The OCT apparatus of this embodiment includes a lateral tracking system (not shown), which allows a scanning beam for OCT to track movement of the eye to be inspected 107 in the XY plane (lateral direction). Accordingly, differently from the embodiments 1 and 2, only the position in the depth direction is determined, and in a method of this embodiment, similarly to the embodiments 1 and 2, the image information by the first irradiation beam 161-a is compared to the reference image information obtained at the step S23. Specifically, a light intensity pattern (gradation pattern) of one piece of the A scan obtained at the step S25 is pattern matched in the depth direction with a light intensity pattern of the reference image information at the corresponding position by using a correlation function. Then, a position that most corresponds is acquired, and a position of one piece of the A scan is identified in the depth direction. This step is applied to all the A scan information.

Next, at step S29, based on the position determination result of the A scan information by the first irradiation beam 161-a in the depth direction at the step S28, a position of the A scan information by the second irradiation beam 161-b is corrected. Because the first irradiation beam 161-a, and the second irradiation beam 161-b are synchronized with each other to scan, a relative positional relation between them is always constant. Therefore, once the position of the A scan information by the first irradiation beam 161-a is determined, a position of the corresponding A scan information by the second irradiation beam 161-b may accordingly be aligned therewith. This step is applied to all the A scan information by the second irradiation beam 161-b. Here, in this embodiment, the lateral tracking system can track movement in the lateral direction (XY directions), so that each position of the corresponding A scan information by the second irradiation beam 161-b can be arranged in scan order to determine each exact position. As described above, the position correction of the tomographic image information corrects a distorted image due to movement of the eye to be inspected 107. Accordingly, also in the OCT apparatus using the Fourier-domain system with a high lateral resolution, blurring in an image due to movement of the eyeball can be easily and more reduced without a complex tracking system operating in the depth direction. In this embodiment, the personal computer 125 controls an on or off of the light source 101, and the light source is turned on only when required. Accordingly, the eye to be inspected 107 does not receive an unnecessary beam, reducing the load on a subject. Note that in this embodiment, the light source 101 is controlled to be turned on or off, but the amount of light may be controlled.

The OCT apparatus for the retina has been described with reference to each of the embodiments described above, but the present invention can be applied to, in addition to it, an OCT apparatus for biological observation directed to a movable object, including observation of the anterior ocular segment and the skin, and observation using an endoscope or a catheter. Also, in each of the embodiments described above, the spectral-domain system, in which coherent light is separated into spectral components, of the OCT apparatus using the Fourier-domain system has been described, but the present invention can be applied to an OCT apparatus using the swept source system in which a light source capable of wavelength scanning is used. Note that, in each embodiment described above, the case where the scan range of the first irradiation beam 161-a, and the scan range of the second irradiation beam 161-b are different, and the case where both scan ranges are consistent with each other have been described, but the scan ranges may be partially overlapped with each other. Further, in each embodiment described above, the information in the depth direction by the first irradiation beam 161-a has been constantly acquired, but the information may be intermittently acquired, or the irradiation may be intermittently conducted with consideration for a difference in lateral resolution between the first irradiation beam 161-a, and the second irradiation beam 161-b. Also, in each embodiment described above, the correlation function has been used for acquiring similarity between the two sets of the A scan information, but other, various evaluation functions may be used.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-052879, filed Mar. 6, 2009, which is hereby incorporated by reference herein in its entirety.

1. An optical tomographic imaging apparatus for imaging a tomographic image of an object by using optical coherence tomography, comprising:
a scanning device for scanning, on the object, a first irradiation beam and a second irradiation beam which are synchronized with each other, in which the first irradiation beam has a spot size larger than that of the second irradiation beam and depth of focus deeper than that of the second irradiation beam;
an image information acquiring device for acquiring first image information obtained with the first irradiation beam and second image information obtained with the second irradiation beam; and
a position correcting device for correcting a position of the second image information by using the position of the first image information that has wider range than the second image information.

2. The optical tomographic imaging apparatus according to claim 1, wherein the scanning device is adapted to be capable of scanning the first irradiation beam and the second irradiation beam closely to each other on the object by using a device for controlling the scan.

3. The optical tomographic imaging apparatus according to claim 1, wherein the reference image information is image information obtained by scanning in advance the first irradiation beam on the object.

4. The optical tomographic imaging apparatus according to claim 1, wherein position correction of the image information obtained by the second irradiation beam is correction in a depth direction corresponding to the Z direction and in a lateral direction corresponding to the XY directions in the XYZ coordinate system.

5. The optical tomographic imaging apparatus according to claim 1,
wherein the position correcting device identifies a position of the first image information based on reference image information, and
wherein based on correlation of a positional relation provided by the synchronized scan between the first image information and the second image information, corrects a position of the second image information by associating the position with the identified position of the first image information.
